(19) United States
(12) Patent Application Publication
(10) Pub. No.: US 2026/0192303 A1
(43) Pub. Date: Jul. 9, 2026

(54) TEST KIT BOX FOR ACCOMMODATING TEST TUBE

(71) Applicant: REBIOGEN CO.,LTD, Gyeonggi-do (KR)

(72) Inventors: Junhyung LEE, Seoul (KR); Seonkyun KIM, Gyeonggi-do (KR)

(21) Appl. No.: 19/380,988

(22) Filed: Nov. 6, 2025

Related U.S. Application Data
(63) Continuation of application No. PCT/KR2025/012784, filed on Aug. 22, 2025.

(30) Foreign Application Priority Data
Jan. 3, 2025 (KR) 10-2025-0000620

(51) Int. Cl. B01L 9/00

(52) U.S. Cl. CPC B01L 9/00; B01L 2300/043; B01L 2300/0609; B01L 2300/0809

(57) ABSTRACT

A test kit box of the present disclosure includes a box plate and a test tube support structure partially separated from the box plate. The structure includes: a grip separated from and provided on the box plate; a test tube lower portion support extending from the grip and folded at least in part under the box plate to support a lower portion; a first test tube side portion support with one portion connected to the test tube lower portion support and an opposite portion connected to the box plate, the opposite portion folded beneath the box plate to support a first side portion; and a second test tube side portion support provided in a side portion of the test tube lower portion support and folded beneath the box plate to support a second side portion opposite the first.

### CROSS-REFERENCE TO RELATED APPLICATION

This application is a U.S. Bypass Continuation Application of International Application No. PCT/KR2025/012784, filed August 22, 2025, which claims priority to and the benefit of Korean Patent Application No. 10-2025-0000620, filed on January 03, 2025, the disclosure of which is incorporated herein by reference in its entirety.

### BACKGROUND

### Technical Field

The present invention relates to a test kit box for accommodating a test tube.

### Background Art

The oral health of a pet is directly related to overall health conditions of the pet, and an oral test plays a crucial role in early detection of diseases. Accordingly, an oral test kit used to collect and analyze saliva of a pet is being introduced. Such a test kit may be used by allowing a user to directly collect saliva of a pet, put the saliva in a test tube, and transport the test tube to a testing facility. However, since a conventional test kit has a high possibility of shaking or breaking while the test tube is transported, there are problems of affecting the reliability of test results and causing inconvenience to a user and a testing facility.

In such a conventional test kit, a separate fixing member is used to secure the test tube. Since the fixing member increases the volume and weight of the test kit, there is a problem of increasing manufacturing and transportation costs. Particularly, when a large quantity of test kits is manufactured and distributed, since an amount of packing material increases, costs are increased, and the environment may be negatively affected.

As a result, there is continuing need for a new structure capable of not only stably protecting a test tube but also minimizing the volume and cost of a test kit.

### Related Art Documents

(Patent Document 1) Japanese Utility Model Laid-Open Publication No. Hei 05-094192 (December 21, 1993)

(Patent Document 2) Korean Registered Patent Publication No. 10-2151267 (September 12, 2022)

### SUMMARY

### Technical Problem

One of the objectives of the present invention is to provide a test kit box capable of stably supporting and protecting a test tube using only a cut and folded structure of a box plate without a separate fixing member to address the limitations of a method of protecting a test tube of a conventional test kit. This is expected to prevent the test tube from shaking or breaking while the test tube is transported, reduce the volume and manufacturing cost of the test kit box, and simplify an assembly process of a test tube support module. The technical objectives of the present invention are not limited to the above-described technical objective, and the other technical objectives which are not described above will be clearly understood to those skilled in the art from the following descriptions.

### Technical Solution

According to some embodiments of the present invention, a test kit box for accommodating a test tube of the present disclosure includes a box plate and a test tube support structure of which at least a portion is separated from the box plate to support a test tube, wherein the test tube support structure includes a grip separated from the box plate and provided on the box plate, a test tube lower portion support extending from a portion of the grip and provided in a state in which at least a portion of the test tube lower portion support is folded under the box plate to support a lower portion of the test tube, a first test tube side portion support of which one portion is connected to the test tube lower portion support, the other portion opposite to the one portion is connected to the box plate, and the other portion is folded beneath the box plate to support a first side portion of the test tube, and a second test tube side portion support provided in a side portion of the test tube lower portion support and folded beneath the box plate to support a second side portion opposite to the first side portion of the test tube.

In the exemplary embodiment, the test tube support structure may further include a test tube lid support provided at one side of the first test tube side portion support and the second test tube side portion support and folded beneath the box plate to support a lid of the test tube and a test tube bottom support provided at the other side opposite to the one side of the first test tube side portion support and the second test tube side portion support and folded beneath the box plate to support a bottom of the test tube.

In the exemplary embodiment, the first test tube side portion support may be cut with respect to the box plate in a “⸦” shape rotated 90º clockwise, the second test tube side portion support may be provided as two second test tube side portion supports at both sides of the test tube lower portion support and cut with respect to the box plate in a “⸦” shape rotated 90º counterclockwise, the test tube lid support may be cut with respect to the box plate in a “⸦” shape inverted in a left-right direction, and the test tube bottom support may be provided to have a smaller size than the test tube lid support and cut with respect to the box plate in a “⸦” shape.

In the exemplary embodiment, the test tube lower portion support and the second test tube side portion support may be cut along a first shared cutting line extending in an up-down direction, the test tube lid support, the first test tube side portion support, and the second test tube side portion support may be cut along a second shared cutting line extending in the up-down direction; the test tube bottom support, the first test tube side portion support, and the second test tube side portion support may be cut along a third shared cutting line extending in the up-down direction, and the first test tube side portion support and the second test tube side portion support may be cut along a fourth shared cutting line extending in the left-right direction.

In the exemplary embodiment, when the test tube is supported by the test tube support structure, the grip may move through a hole formed by separating the test tube lower portion support in a state in which at least a portion of the grip is supported on the box plate.

In the exemplary embodiment, the test kit box for accommodating a test tube may further include a catch extending from a folding line of the second test tube side portion support and separated to protrude upward from the box plate to block movement of the grip.

A thickness of each of the grip, the test tube lower portion support, the first test tube side portion support, the second test tube side portion support, the test tube lid support, and the test tube bottom support may be the same as a thickness of the box plate.

### Advantageous Effects

According to embodiments of the present invention, the limitations of a method of using a separate fixing member for protecting a test tube in a conventional test kit can be addressed to provide a test kit box capable of stably supporting and protecting a test tube using only a cut and folded structure of a box plate. Through this, there are effects of preventing the test tube from shaking and breaking while the test tube is transported, reducing the volume and manufacturing cost of the test kit box, and simplifying an assembly process.

The technical effects according to the embodiments of the present invention are not limited to the above-described effects, and the other effects which are not described above will be clearly understood to those skilled in the art from descriptions of the present specification.

### BRIEF DESCRIPTION OF THE DRAWINGS

FIG. 1 is an assembly view of a test kit box according to an exemplary embodiment of the present disclosure.

FIG. 2 is a development view of the test kit box according to the exemplary embodiment of the present disclosure.

FIG. 3 is an enlarged view of a test tube support structure of the test kit box according to the exemplary embodiment of the present disclosure.

FIG. 4 is a view illustrating the test tube support structure separated from a box plate according to the exemplary embodiment of the present disclosure.

FIG. 5 is a view illustrating a state in which the test tube support structure according to the exemplary embodiment of the present disclosure supports a test tube on the box plate.

FIG. 6 is a view illustrating a state in which the test tube support structure according to the exemplary embodiment of the present disclosure supports the test tube under the box plate.

FIG. 7 is a view illustrating a process in which the test tube is separated from the box plate according to the exemplary embodiment of the present disclosure.

FIG. 8 is a perspective view illustrating the test kit box according to the exemplary embodiment of the present disclosure.

### DETAILED DESCRIPTION

A test kit box for accommodating a test tube of the present disclosure includes a box plate and a test tube support structure of which at least a portion is separated from the box plate to support a test tube, wherein the test tube support structure includes a grip separated from the box plate and provided on the box plate, a test tube lower portion support extending from a portion of the grip and provided in a state in which at least a portion of the test tube lower portion support is folded under the box plate to support a lower portion of the test tube, a first test tube side portion support of which one portion is connected to the test tube lower portion support, the other portion opposite to the one portion is connected to the box plate, and the other portion is folded beneath the box plate to support a first side portion of the test tube, a second test tube side portion support provided in a side portion of the test tube lower portion support and folded beneath the box plate to support a second side portion opposite to the first side portion of the test tube, and a catch which extends from a folding line of the second test tube side portion support, is separated from the box plate to protrude upward from the box plate, and is formed to block movement of the grip, the grip is formed to move on the box plate through a hole formed by separating the test tube lower portion support when the test tube is coupled to the test tube support structure, and the catch restricts movement of the grip to prevent the grip from passing through the hole formed by separating the lower portion support and moving to a hole formed by separating the second test tube side portion support when the test tube is coupled to the test tube support structure.

### Modes of the Invention

Hereinafter, embodiments of the present invention will be described in detail with reference to the accompanying drawings. The following description is only provided to embody the embodiments and does not restrict or limit the scope of the present invention. Content that may be easily construed from the detailed description and the embodiments of the invention by those skilled in the art related to the present invention should be interpreted as being included in the scope of the present invention. Detailed descriptions of contents widely known to the skilled in the art related to the present invention will be omitted.

Although terms used in the present invention are used among general terms widely used in the art related to the present invention, meanings of the terms used in the present invention may be changed according to intentions of those skilled in the art, the advent of new technologies, examination standards, precedents, or the like. Some terms may be arbitrary selected by the applicants, and in this case, meanings thereof will be described in detail. The terms used in the present invention should be interpreted not only with their dictionary meanings but also with meanings reflecting the overall context of the specification.

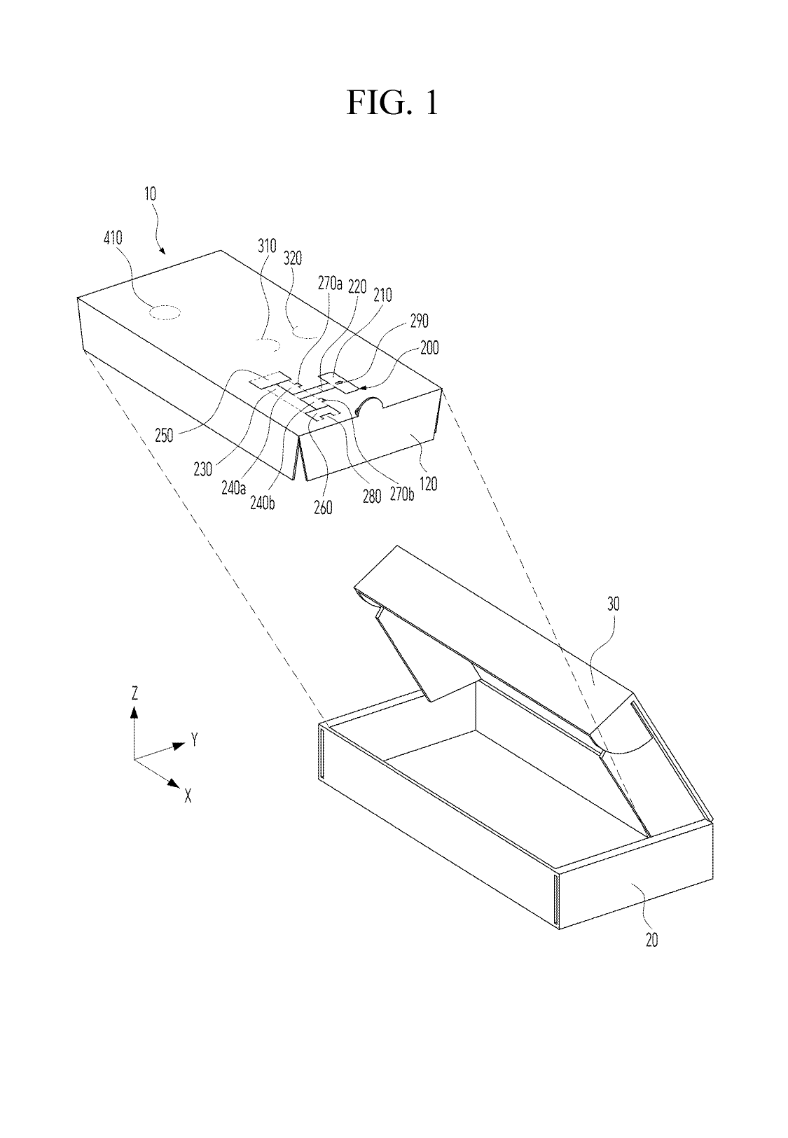

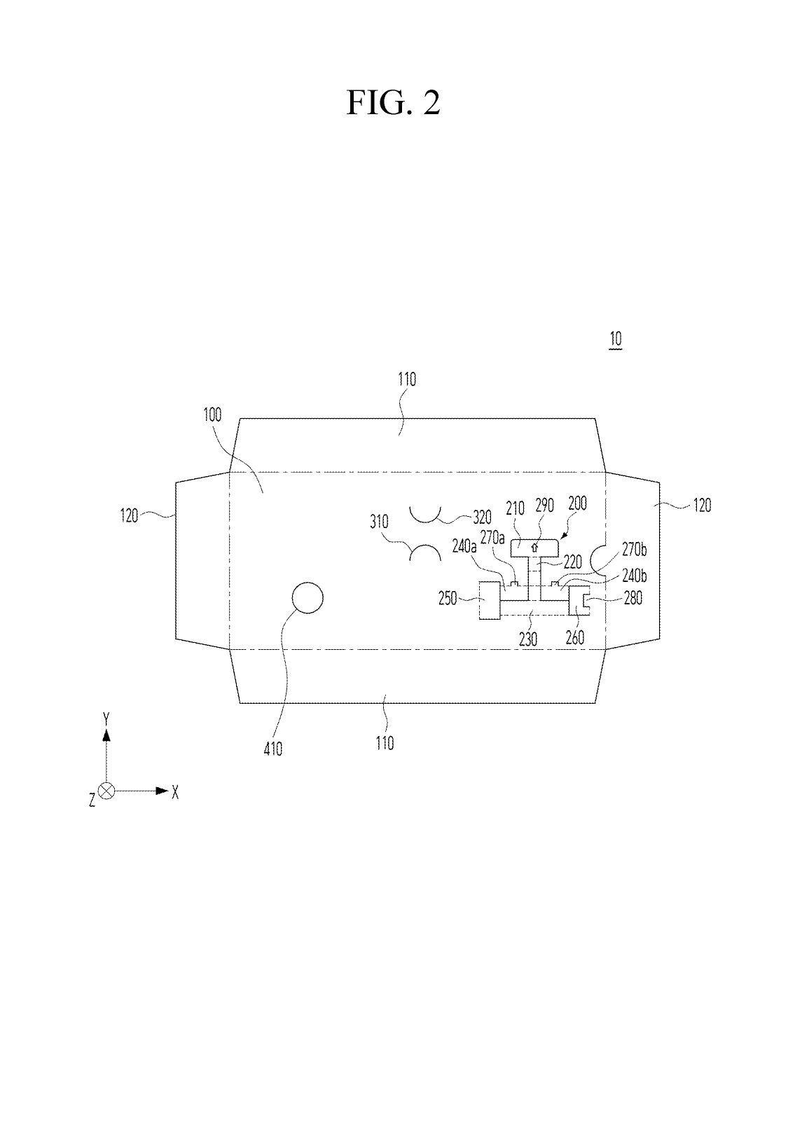

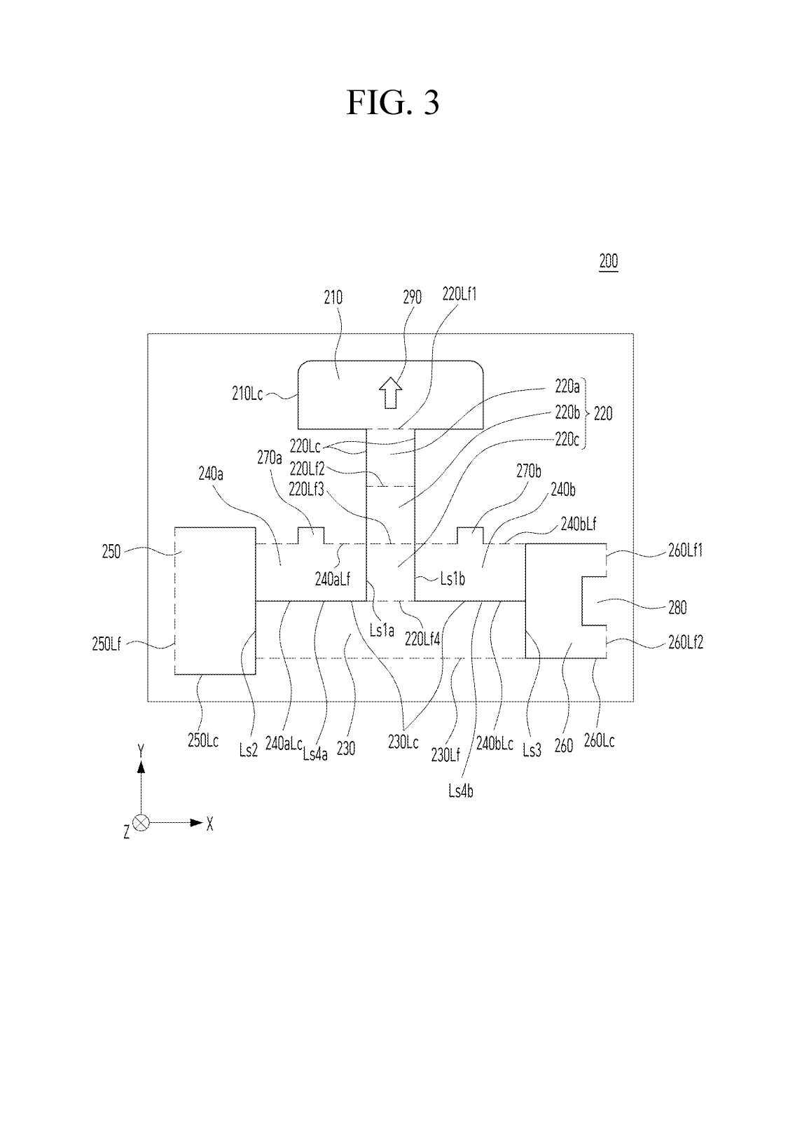

FIG. 1 is an assembly view of a test kit box 10 according to an exemplary embodiment of the present disclosure. FIG. 2 is a development view of the test kit box 10 according to the exemplary embodiment of the present disclosure. In addition, FIG. 3 is an enlarged view of a test tube support structure 200 of the test kit box 10 according to the exemplary embodiment of the present disclosure.

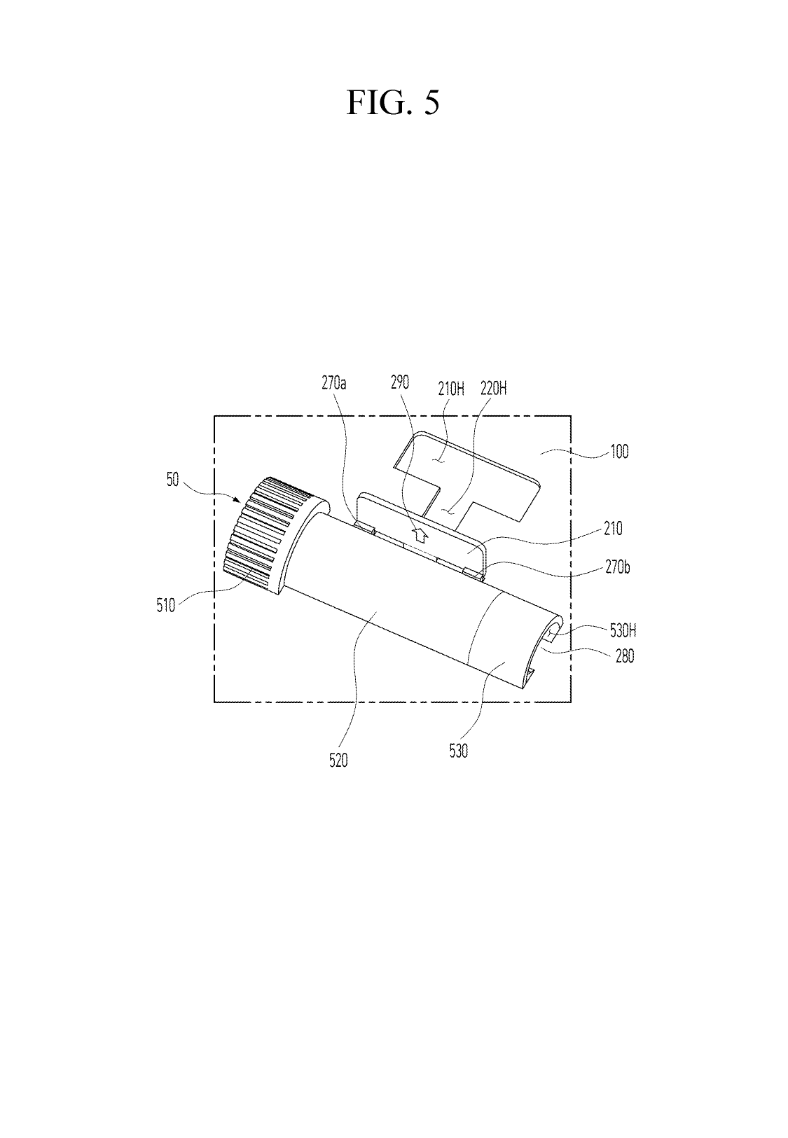

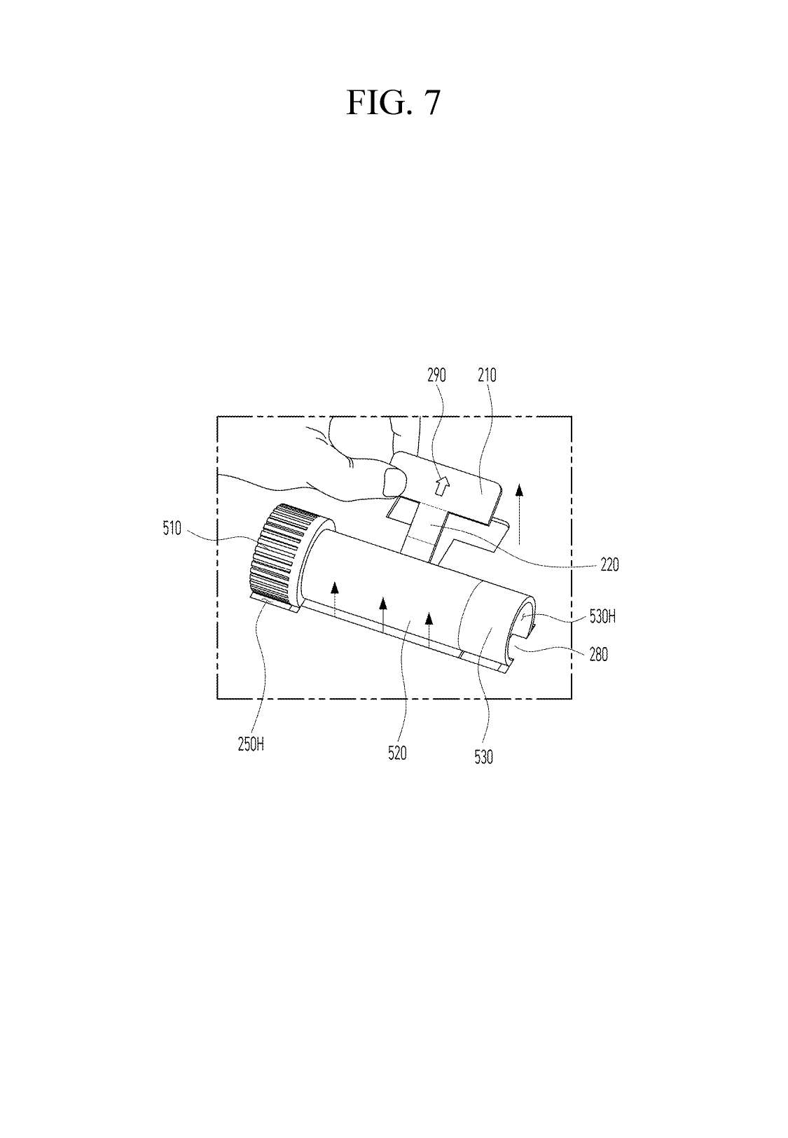

The test kit box 10 of the present disclosure may be a box which is accommodated in an inner space of a case 20. In addition, the test kit box 10 may be accommodated in the inner space of the case 20 and closed by a lid 30 connected to the case 20. That is, the test kit box 10 of the present disclosure may be a box which accommodates a test swab CS (see FIG. 7) and a test tube 50 (see FIG. 5) in the case 20.

In addition, the test kit box 10 of the present invention may include the test tube support structure 200 which is formed by cutting and folding at least a portion of the test kit box 10, is separated from a box plate 100, and supports the test tube 50.

In the exemplary embodiment, the test kit box 10 of the present disclosure may be a box for an oral health test of a pet and may serve to safely transport the test tube 50 including saliva of the pet to a testing facility. However, the role of the test kit box 10 of the present disclosure is not limited to the above-described role.

Hereinafter, the test tube support structure 200 of the test kit box 10 of the present disclosure will be described in detail with references to FIGS. 1 to 3.

The test kit box 10 of the present disclosure may include the box plate 100, the test tube support structure 200, test swab support structures 310 and 320, a test tube insertion member 410, and the like.

The box plate 100 may be a plate which is a base of the test kit box 10 of the present disclosure. In the exemplary embodiment, the box plate 100 may be provided in a rectangular shape, but the shape of the box plate 100 is not limited to the above-described shape.

In addition, a first side plate 110 may be provided at one side of the box plate 100, and a second side plate 120 may be provided at the other side of the box plate 100. As the first side plate 110 and the second side plate 120 are folded with respect to the box plate 100, the test kit box 10 of the present disclosure may be provided in a rectangular parallelepiped shape of which a bottom surface is open and accommodated in the inner space of the case 20.

In the exemplary embodiment, the box plate 100 may be a plate which provides the test tube support structure 200, the test swab support structures 310 and 320, and the test tube insertion member 410. Specifically, when the test kit box 10 of the present disclosure is provided to a user, the test tube support structure 200, the test swab support structures 310 and 320, and the test tube insertion member 410 of the present disclosure may be integrally provided with the box plate 100. For example, the test tube support structure 200, the test swab support structures 310 and 320, and the test tube insertion member 410 of the present disclosure may be disposed to be coplanar with the box plate 100.

That is, when the test kit box 10 of the present disclosure is provided to the user, a thickness of each of the test tube support structure 200, the test swab support structures 310 and 320, and the test tube insertion member 410 of the present disclosure may be substantially the same as a thickness of the box plate 100.

In the exemplary embodiment, the material of the box plate 100 may be provided as paper. Accordingly, the first side plate 110 and the second side plate 120 of the box plate 100 may be easily folded. In addition, since the material of the test tube support structure 200 may also be provided as paper, the test tube support structure 200 may be easily cut or folded with respect to the box plate 100.

In the exemplary embodiment, the test tube support structure 200, the test swab support structures 310 and 320, and the test tube insertion member 410 of the present disclosure may be implemented in a three-dimensional shape by being cut or folded with respect to the box plate 100.

Hereinafter, when the box plate 100 is viewed from above, a direction in which a relatively long side of the box plate 100 extends may be defined as a left-right direction (X direction), a direction in which a relatively short side thereof extends may be defined as an up-down direction (Y direction), and a direction perpendicular to the left-right direction and the up-down direction may be defined as a thickness direction (Z direction).

In addition, in FIG. 3, a solid line indicates a cutting line along which the test tube support structure 200 of the present disclosure is separated from the box plate 100, and a dotted line indicates a line along which the test tube support structure 200 of the present disclosure is folded with respect to the box plate 100.

The test tube support structure 200 of the present disclosure may be three-dimensionally formed by being cut and folded with respect to the box plate 100 in the thickness direction (Z direction) to support and protect the test tube 50. Specifically, the test tube support structure 200 of the present disclosure may be formed to stably support and protect the test tube 50 to safely transport the test tube 50 including the saliva of the pet to the testing facility.

The test tube support structure 200 of the present disclosure may include a grip 210, a test tube lower portion support 220, a first test tube side portion support 230, second test tube side portion supports 240a and 240b, a test tube lid support 250, a test tube bottom support 260, catches 270a and 270b, a bottom fixing portion 280, and the like.

Since the test tube support structure 200 of the present disclosure may include the grip 210, the test tube lower portion support 220, the first test tube side portion support 230, the second test tube side portion supports 240a and 240b, the test tube lid support 250, the test tube bottom support 260, the catches 270a and 270b, and the bottom fixing portion 280, the test tube support structure 200 may prevent the test tube 50 from shaking and breaking by supporting a lid, a bottom, a side portion of the test tube 50 in all directions while the test tube 50 is transported.

Particularly, since a plurality of supports 210 to 280 which are described above are formed by being cut and folded with respect to the box plate 100, the test tube 50 can be stably fixed without a separate part. Through this, a protection function for the test tube 50 can be improved to improve the reliability of test results.

Hereinafter, the structure and function of the test tube support structure 200 of the present disclosure will be described in more detail.

The grip 210 may be a portion of the test tube support structure 200 provided for the user to grip when the test tube 50 is separated from the box plate 100.

In the exemplary embodiment, the grip 210 may be provided to be cut with respect to the box plate 100 and to protrude upward from the box plate 100. That is, when the test tube 50 is coupled to the test tube support structure 200, the grip 210 may protrude from an upper portion of the box plate 100 in the thickness direction (Z direction) such that the user easily grips the grip 210.

In the exemplary embodiment, the grip 210 may be provided in a rectangular shape. For example, the grip 210 may be provided in the rectangular shape of which a length in the left-right direction (X direction) is greater than a length in the up-down direction (Y direction). However, the shape of the grip 210 is not limited to the above-described shape and may be provided in any of various shapes. For example, the grip 210 may be provided in a circular shape, a polygonal shape, or the like.

In the exemplary embodiment, a grip cutting line 210Lc may be provided along the periphery of the grip 210. The grip cutting line 210Lc may have a cutting groove to easily perform a process of cutting the grip. That is, the grip cutting line 210Lc may be a cutting groove having a shape in which a portion of the box plate 100 is concavely recessed.

In addition, a portion of the grip 210 may be connected to the test tube lower portion support 220 which will be described below, and a first lower portion support folding line 220Lf1may be provided between the grip 210 and the test tube lower portion support 220. The grip 210 may be provided to be bent from the test tube lower portion support 220 while folded along the first lower portion support folding line 220Lf1.

The test tube lower portion support 220 may be a portion of the test tube support structure 200 formed to support a lower portion of the test tube 50 when the test tube 50 is coupled to the test tube support structure 200. The lower portion of the test tube 50 may be defined as the lowest portion of the test tube 50 located in the thickness direction (Z direction) when a lid 510 (see FIG. 5) and a bottom 530 (see FIG. 5) of the test tube 50 are coupled to the test tube support structure 200 to face the left-right direction (X direction).

In the exemplary embodiment, the test tube lower portion support 220 may be provided to protrude from a lower portion of the box plate 100 in a thickness direction (-Z direction) by being cut with respect to the box plate 100.

In addition, the test tube lower portion support 220 may be folded along the first lower portion support folding line 220Lf1, a second lower portion support folding line 220Lf2 provided under the first lower portion support folding line 220Lf1, a third lower portion support folding line 220Lf3 provided under the second lower portion support folding line 220Lf2, and a fourth lower portion support folding line 220Lf4 provided under the third lower portion support folding line 220Lf3.

Accordingly, the test tube lower portion support 220 may include a first test tube lower portion support 220a folded to be bent from the grip 210 and provided between the first lower portion support folding line 220Lf1 and the second lower portion support folding line 220Lf2, a second test tube lower portion support 220b folded to be bent from the first test tube lower portion support 220a and the third test tube lower portion support 220c, and a third test tube lower portion support 220c folded to be bent from the first test tube side portion support 230 and provided between the third lower portion support folding line 220Lf3 and the fourth lower portion support folding line 220Lf4.

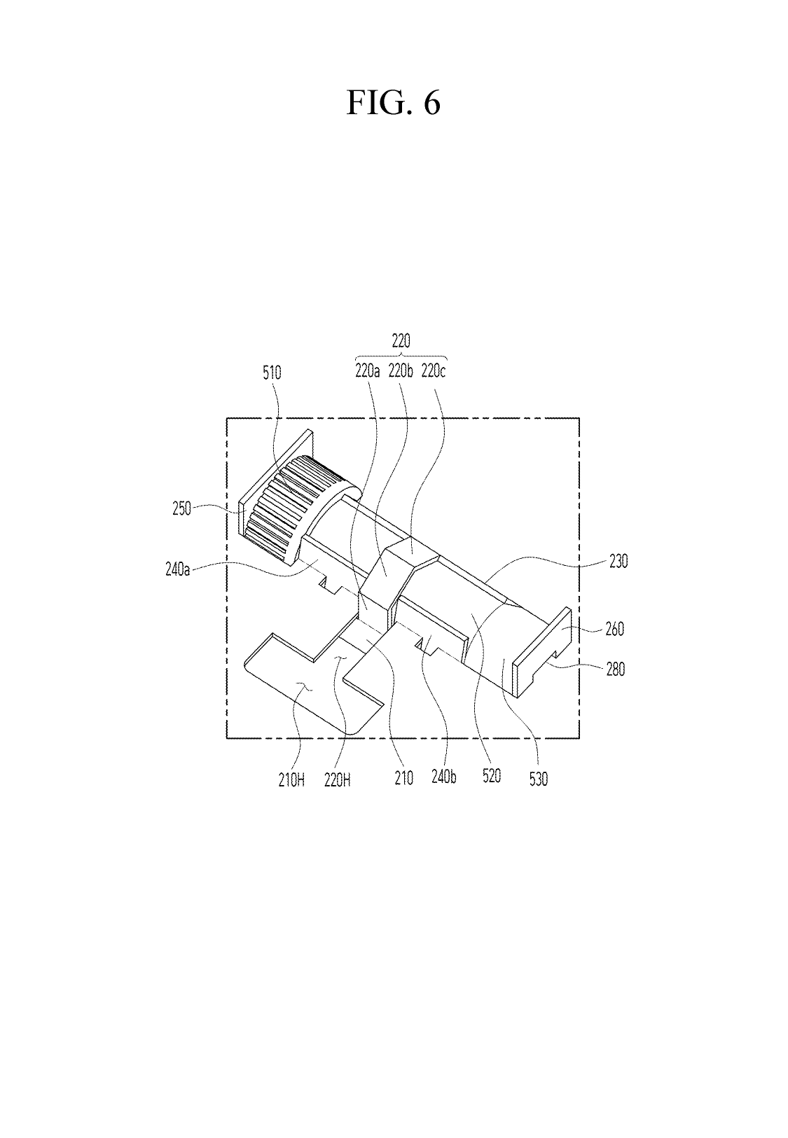

As the test tube lower portion support 220 of the present disclosure is folded along the first to fourth lower portion support folding lines 220Lf1 to 220Lf4, the test tube lower portion support 220 may be provided in a shape similar to a “U” shape or convex bowl shape to protrude from the lower portion of the box plate 100 in the thickness direction as described in FIG. 6.

In the exemplary embodiment, separation distances between the first to fourth lower portion support folding lines 220Lf1 to 220Lf4 may be provided to be the same. In addition, the first lower portion support folding line 220Lf1 may be provided to be substantially collinear with the cutting line 210Lc of the grip 210, the third lower portion support folding line 220Lf3 may be provided to be substantially collinear with folding lines 240aLf and 240bLf of the second test tube side portion supports 240a and 240b, and the fourth lower portion support folding line 220Lf4 may be provided to be substantially collinear with a cutting line 230Lc of the first test tube side portion support 230.

In addition, each of the first to fourth lower portion support folding lines 220Lf1 to 220Lf4 may be provided in the shape of a line extending in the left-right direction (X direction).

In the exemplary embodiment, as the test tube 50 is provided in a cylindrical shape, when the test tube 50 is coupled to the test tube support structure 200, the lower portion of the test tube 50 may form a curved surface. Since the test tube lower portion support 220 of the present disclosure may be provided in the “U” shape or bowl shape which is convex downward by being folded along the first to fourth lower portion support folding lines 220Lf1 to 220Lf4 multiple times, a contact area between the test tube lower portion support 220 and the lower portion of the test tube 50 increases so that the test tube lower portion support 220 can stably support the lower portion of the test tube 50.

In the exemplary embodiment, the test tube lower portion support 220 may be provided in a rectangular shape of which a length in the left-right direction (X direction) is smaller than a length in the up-down direction (Y direction). In addition, as an upper portion of the test tube lower portion support 220 is connected to the grip 210, and the lower portion thereof is connected to the first test tube side portion support 230, a cutting line 220Lc of the test tube lower portion support 220 may be provided in the up-down direction (Y direction).

In addition, a portion of the cutting line 220Lc of the test tube lower portion support 220 may overlap portions of cutting lines 240aLc and 240bLc of the second test tube side portion supports 240a and 240b. The overlapping cutting lines may be defined as first shared cutting lines Ls1a and Ls1b, and the test tube lower portion support 220 and the second test tube side portion supports 240a and 240b may be cut along the first shared cutting lines Ls1a and Ls1b extending vertically.

The first test tube side portion support 230 may be provided to protrude from the lower portion of the box plate 100 in the thickness direction (-Z direction) by being cut and folded with respect to the box plate 100. Specifically, the first test tube side portion support 230 may be provided to protrude in the thickness direction (-Z direction) by being cut with respect to the box plate 100 in a “⸦” shape rotated 90º clockwise.

In addition, the first test tube side portion support 230 may be a portion of the test tube support structure 200 formed to support a first side portion of the test tube 50, when the test tube 50 is coupled to the test tube support structure 200. The first side portion of the test tube 50 may be the lowest portion of the test tube 50 located in a downward direction (-Y direction) of the box plate 100 when the lid 510 and the bottom 530 of the test tube 50 are coupled to the test tube support structure 200 to face the left-right direction (X direction).

In the exemplary embodiment, an upper portion and a side portion of the first test tube side portion support 230 may be cut with respect to the box plate 100. Specifically, the cutting line 230Lc of the first test tube side portion support 230 may be provided along the upper portion and the side portion of the first test tube side portion support 230, the cutting line 230Lc of the upper portion may be collinear with the cutting lines 240aLc and 240bLc of the second test tube side portion supports 240a and 240b, the cutting line 230Lc of a left side portion may be collinear with a cutting line 250Lc of the test tube lid support 250, and the cutting line 230Lc of a right side portion may be collinear with a cutting line 260Lc of the test tube bottom support 260.

In the exemplary embodiment, a lower portion of the first test tube side portion support 230 may be folded with respect to the box plate 100. Specifically, the first test tube side portion support 230 may be provided to protrude in the thickness direction (-Z direction) by being folded with respect to the box plate 100 based on a folding line 230Lf. Accordingly, the first test tube side portion support 230 of the present disclosure may support the first side portion of the test tube 50.

In the exemplary embodiment, the first test tube side portion support 230 may be provided in a rectangular shape of which a length in the left-right direction (X direction) is greater than a length in the up-down direction (Y direction). For example, the length of the first test tube side portion support 230 in the left-right direction (X direction) may be provided to be greater than a length of the grip 210 in the left-right direction (X direction).

In addition, the length of the first test tube side portion support 230 in the up-down direction (Y direction) may be substantially the same as a length of each of the second test tube side portion supports 240a and 240b in the up-down direction (Y direction) which will be described below. In addition, the side portion cutting line 230Lc of the first test tube side portion support 230 may be substantially collinear with the side portion cutting lines 240aLc and 240bLc of the second test tube side portion supports 240a and 240b.

That is, protruding lengths of the first test tube side portion support 230 and the second test tube side portion supports 240a and 240b in the thickness direction (-Z direction) may be substantially the same, and lengths (that is, widths) of the first test tube side portion support 230 and the second test tube side portion supports 240a and 240b in the left-right direction (X direction) may be substantially the same. Accordingly, the first test tube side portion support 230 and the second test tube side portion supports 240a and 240b of the present disclosure may stably support the side portion of the test tube 50 using a uniform force.

In the exemplary embodiment, a left cutting line 240Lc of the first test tube side portion support 230 may overlap a portion of the cutting line 250Lc of the test tube lid support 250. The overlapping cutting line may be defined as a second shared cutting line Ls2, and the first test tube side portion support 230 and the test tube lid support 250 may be cut along the second shared cutting line LS2 extending in the up-down direction (Y direction).

In addition, a right cutting line 240Lc of the first test tube side portion support 230 may overlap a portion of the cutting line 260Lc of the test tube bottom support 260. The overlapping cutting line may be defined as a third shared cutting line Ls3, and the first test tube side portion support 230 and the test tube bottom support 260 may be cut along the third shared cutting line Ls3 extending in the up-down direction (Y direction).

In addition, an upper cutting line 240Lc of the first test tube side portion support 230 may overlap portions of the cutting lines 240aLc and 240bLc of the second test tube side portion supports 240a and 240b. The overlapping cutting lines may be defined as fourth shared cutting lines Ls4a and Ls4b, and the first test tube side portion support 230 and the second test tube side portion supports 240a and 240b may be cut along the fourth shared cutting lines Ls4a and Ls4b extending in the left-right direction (X direction).

As the second test tube side portion supports 240a and 240b are provided at side portions of the test tube lower portion support 220 and folded beneath the box plate 100, the second test tube side portion supports 240a and 240b may be formed to support the side portion of the test tube 50.

Specifically, the second test tube side portion supports 240a and 230b may be provided as two portions at both side portions of the test tube lower portion support 220 and cut with respect to the box plate 100 in a “⸦” shape rotated 90º counterclockwise.

In addition, the second test tube side portion supports 240a and 240b may be provided to protrude from the lower portion of the box plate 100 in the thickness direction (-Z direction) by being cut and folded with respect to the box plate 100 and may face the first test tube side portion support 230.

In addition, the second test tube side portion supports 240a and 240b may be portions of the test tube support structure 200 formed to support a second side portion of the test tube 50 when the test tube 50 is coupled to the test tube support structure 200. The second side portion of the test tube 50 may be the highest portion of the test tube 50 located in an upward direction (+Y direction) of the box plate 100 when the lid 510 and the bottom 530 of the test tube 50 are coupled to the test tube support structure 200 to face the left-right direction (X direction).

In the exemplary embodiment, lower portions and side portions of the second test tube side portion supports 240a and 240b may be cut with respect to the box plate 100. Specifically, the cutting lines 240aLc and 240bLc of the second test tube side portion supports 240a and 240b may be provided along the lower portions and the side portions of the second test tube side portion supports 240a and 240b.

In the exemplary embodiment, a left cutting line of the cutting line 240aLc of the second test tube side portion support 240a provided at a left side may be substantially collinear with the cutting line 250Lc of the test tube lid support 250, a right cutting line thereof may be substantially collinear with the cutting line 220Lc of the test tube lower portion support 220, and a lower cutting line thereof may be substantially collinear the cutting line 230Lc of the first test tube side portion support 230.

In the exemplary embodiment, a left cutting line of the cutting line 240bLc of the second test tube side portion support 240b provided at a right side may be substantially collinear with the cutting line 220Lc of the test tube lower portion support 220, a right cutting line thereof may be substantially collinear with the cutting line 260Lc of the test tube bottom support 260, and a lower cutting line thereof may be substantially collinear with the cutting line 230Lc of the first test tube side portion support 230.

In the exemplary embodiment, upper portions of the second test tube side portion supports 240a and 240b may be folded with respect to the box plate 100. Specifically, the second test tube side portion supports 240a and 240b may be provided to be folded with respect to the box plate 100 to protrude in the thickness direction (-Z direction) based on the folding lines 240aLf and 240bLf. Accordingly, the second test tube side portion supports 240a and 240b of the present disclosure may support the second side portion of the test tube 50.

In the exemplary embodiment, each of the second test tube side portion supports 240a and 240b may be provided in a rectangular shape of which the length in the left-right direction (X direction) is greater than the length in the up-down direction (Y direction).

In the exemplary embodiment, the upper portions of the second test tube side portion supports 240a and 240b may be connected to the catches 270a and 270b which will be described below. For example, as the second test tube side portion supports 240a and 240b are provided as two portions, the catches 270a and 270b may also be provided as two portions.

As the test tube lid support 250 is provided at one side of the first test tube side portion support 230 and the second test tube side portion support 240a and folded beneath the box plate 100, the test tube lid support 250 may be formed to protrude in the thickness direction (-Z direction) to support the lid 510 of the test tube 50. Specifically, the test tube lid support 250 may be provided to protrude in the thickness direction (-Z direction) by being cut with respect to the box plate 100 in a “⸦” shape inverted in the left-right direction.

In addition, the test tube lid support 250 may be a portion of the test tube support structure 200 formed to support the lid 510 of the test tube 50 when the test tube 50 is coupled to the test tube support structure 200. The lid 510 of the test tube 50 may serve to seal contents in the test tube and may be the farthest portion of the test tube 50 located in a leftward direction (-X direction) of the box plate 100 when the lid 510 and the bottom 530 of the test tube 50 are coupled to the test tube support structure 200 to face the left-right direction (X direction).

In the exemplary embodiment, an upper portion, a lower portion, and a right side portion of the test tube lid support 250 may be cut with respect to the box plate 100 based on the cutting line 250Lc. In addition, a left side portion of the test tube lid support 250 may be folded with respect to the box plate 100 along a folding line 250Lf.

In the exemplary embodiment, the test tube lid support 250 may be provided in a rectangular shape of which a length in the up-down direction (Y direction) is greater than a length in the left-right direction (X direction). For example, the length of the test tube lid support 250 in the up-down direction (Y direction) may be provided to be greater than the sum of the lengths of the first test tube side portion support 230 and the second test tube side portion supports 240a and 240b, which are described above, in the up-down direction (Y direction). This is to reflect that a width of the lid 510 is greater than that of a main body of the test tube 50. The test tube lid support 250 of the present disclosure may protrude in the thickness direction (-Z direction) to be in close contact with the lid 510 of the test tube 50.

As the test tube bottom support 260 is provided at the other side of the first test tube side portion support 230 and the second test tube side portion support 240b and folded beneath the box plate 100, the test tube bottom support 260 may be formed to protrude in the thickness direction (-Z direction) to support the bottom 530 of the test tube 50. Specifically, the test tube bottom support 260 may be provided to protrude in the thickness direction (-Z direction) by being cut with respect to the box plate 100 in a “⸦” shape.

In addition, the test tube bottom support 260 may be a portion of the test tube support structure 200 formed to support the bottom 530 of the test tube 50 when the test tube 50 is coupled to the test tube support structure 200. The bottom 530 of the test tube 50 may be a bottom portion of a space in which the contents of the test tube is accommodated and may be the farthest portion of the test tube 50 located in a rightward direction (+X direction) of the box plate 100 when the lid 510 and the bottom 530 of the test tube 50 are coupled to the test tube support structure 200 to face the left-right direction (X direction).

In the exemplary embodiment, an upper portion, the lower portion, and a left side portion of the test tube bottom support 260 may be cut with respect to the box plate 100 based on the cutting line 260Lc. In addition, a right side portion of the test tube bottom support 260 may be folded with respect to the box plate 100 based on folding lines 260Lf1 and 260Lf2. The folding lines 260Lf1 and 260Lf2 may be provided as two lines and disposed to be spaced apart from each other by the bottom fixing portion 280, which will be described below, in the up-down direction.

In the exemplary embodiment, the test tube bottom support 260 may be provided in a rectangular shape of which a length in the up-down direction (Y direction) is greater than a length in the left-right direction (X direction). For example, the length of the test tube bottom support 260 in the up-down direction (Y direction) may be substantially the same as the sum of the lengths of the first test tube side portion support 230 and the second test tube side portion supports 240a and 240b, which are described above, in the up-down direction (Y direction). This is to reflect that the main body has a constant width in a longitudinal direction in the test tube 50. The test tube bottom support 260 of the present disclosure may protrude in the thickness direction (-Z direction) to be in close contact with the bottom 530 of the test tube 50.

In addition, a side portion of the test tube bottom support 260 may be connected to the bottom fixing portion 280 which will be described below. For example, the bottom fixing portion 280 may be formed to protrude leftward from the folding line 260Lf1 of the test tube bottom support 260.

The catches 270a and 270b may extend from the folding lines 240aLf and 240bLf of the second test tube side portion supports 240a and 240b and may be separated to protrude from one surface of the box plate 100 in a thickness direction (+Z direction). In addition, the catches 270a and 270b may protrude from the box plate 100 to block the movement of the grip 210 in the downward direction (-Y direction).

In the exemplary embodiment, as the second test tube side portion supports 240a and 240b are provided as two portions, the catches 270a and 270b of the present disclosure may also be formed as two portions. In addition, each of the catches 270a and 270b may protrude from the upper portion of the box plate 100 by being provided in a “⸦” shape rotated 90º clockwise.

In the exemplary embodiment, as the catches 270a and 270b extend from the folding lines 240aLf and 240bLf of the second test tube side portion supports 240a and 240b, when the second test tube side portion supports 240a and 240b are folded in the thickness direction (-Z direction), the catches 270a and 270b may be provided to protrude in the thickness direction (+Z direction) of the box plate 100.

In the exemplary embodiment, each of the catches 270a and 270b may have a rectangular shape, and in this case, a length thereof in the left-right direction (X direction) may be provided to be greater than a length in the up-down direction (Y direction). In addition, the catches 270a and 270b may be disposed at locations overlapping a space formed by the grip 210 in the left-right direction (X direction) such that the catches 270a and 270b block the movement of the grip 210.

In the exemplary embodiment, since the catches 270a and 270b may be separated to protrude upward from the box plate 100, the catches 270a and 270b may support the side portion of the test tube 50 on the box plate 100.

The bottom fixing portion 280 may extend from the folding lines 260Lf1 and 260Lf2 of the test tube bottom support 260 and protrude in the leftward direction (-X direction) of the box plate 100. In addition, the bottom fixing portion 280 may be formed to be inserted into a groove 530H (see FIG. 5) provided in the bottom 530 of the test tube 50. Accordingly, the bottom fixing portion 280 may safely fix the bottom 530 of the test tube 50.

In the exemplary embodiment, the bottom fixing portion 280 may be provided in a “⸦” shape inverted in the left-right direction and may protrude from the folding lines 260Lf1 and 260Lf2 of the test tube bottom support 260.

In the exemplary embodiment, the bottom fixing portion 280 may have a rectangular shape, and in this case, a length thereof in the up-down direction (Y direction) may be provided to be greater than a length thereof in the left-right direction (X direction).

In the exemplary embodiment, an outer side of the test tube lid support 250, the first test tube side portion support 230, the second test tube side portion supports 240a and 240b, and the test tube bottom support 260 may be substantially the same as or similar to an outer shape of the test tube 50.

In addition, the grip 210 of the present disclosure may further include an indication maker 290 indicating a separation direction for separating the test tube 50. In the exemplary embodiment, the indication maker 290 may be provided in an arrow shape which guides a pulling direction of the grip 210. Since the indication maker 290 may guide the pulling direction of the grip 210 to the user, the user can smoothy separate the test tube 50.

Since the test tube support structure 200 of the present disclosure may include the test tube lower portion support 220, the first test tube side portion support 230, the second test tube side portion supports 240a and 240b, the test tube lid support 250, and the test tube bottom support 260, the test tube support structure 200 of the present disclosure may support all of the lid, the bottom, a lower surface, and a side surface of the test tube 50. Accordingly, the test tube 50 can be prevented from shaking or breaking while transported.

In addition, since a plurality of such supports are formed through cutting and folding the test kit box 10, the manufacturing cost of the test kit box 10 of the present disclosure can be reduced, and an assembly process thereof can be simplified.

In addition, as the test tube support structure 200 of the present disclosure includes the grip 210 protruding from the box plate 100, the user may pull the grip 210 to easily separate the test tube 50 from the test kit box 10.

In addition, since the test tube support structure 200 of the present disclosure may include the catches 270a and 270b which restrict the movement of the grip 210 in the downward direction (-Y direction), when the test tube 50 is coupled to the test tube support structure 200, the structural safety of the test tube support structure 200 can be secured.

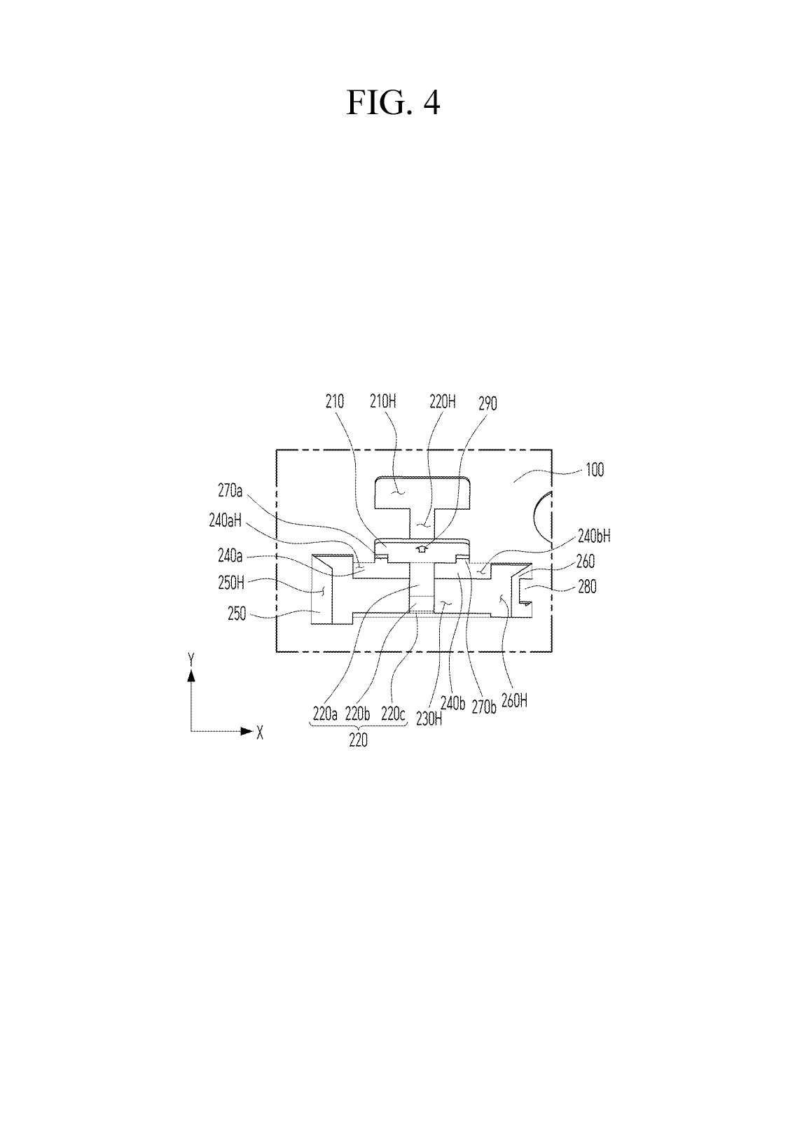

FIG. 4 is a view illustrating the test tube support structure 200 separated from the box plate 100 according to the exemplary embodiment of the present disclosure, FIG. 5 is a view illustrating a state in which the test tube support structure 200 according to the exemplary embodiment of the present disclosure supports the test tube 50 on the box plate 100, and FIG. 6 is a view illustrating a state in which the test tube support structure 200 according to the exemplary embodiment of the present disclosure supports the test tube 50 under the box plate 100.

Referring to FIGS. 4 to 6, as the test tube support structure 200 of the present disclosure is separated from the box plate 100, a grip hole 210H may be formed in a portion from which the grip 210 is separated, a lower portion support hole 220H may be formed in a portion from which the test tube lower portion support 220 is separated, a first side portion support hole 230H may be formed in a portion from which the first test tube side portion support 230 is separated, second side portion support holes 240aH and 240bH may be formed in portions from which the second test tube side portion supports 240a and 240b are separated, a lid support hole 250H may be formed in a portion from which the test tube lid support 250 is separated, and a bottom support hole 260H may be formed in a portion from which the test tube bottom support 260 is separated.

The test tube 50 may be inserted into a space provided by the lower portion support hole 220H, the first side portion support hole 230H, the second side portion support holes 240aH and 240bH, the lid support hole 250H, and the bottom support hole 260H. A shape of the combined holes may be substantially the same as or similar to a shape of an exterior of the test tube 50, and a size of the combined holes may also be substantially the same as or similar to a size of the test tube 50.

In addition, the lid 510 of the test tube 50 may be supported by the test tube lid support 250, the bottom 530 of the test tube 50 may be supported by the test tube bottom support 260, one portion of a side portion 520 of the test tube 50 may be supported by the first test tube side portion support 230, and the other portion of the side portion 520 of the test tube 50 may be supported by the second test tube side portion supports 240a and 240b, and the lower portion of the test tube 50 may be supported by the test tube lower portion support 220. That is, the lid, the bottom, the lower portion, and the side portion of the test tube 50 may be firmly supported by the plurality of supports.

In the exemplary embodiment, the grip 210 of the present disclosure may be formed to move in the up-down direction along the lower portion support hole 220H formed as the test tube lower portion support 220 is separated therefrom.

In addition, as the catches 270a and 270b of the present disclosure protrude from the box plate 100 in the thickness direction (+Z direction), the catches 270a and 270b may support a portion of the side portion of the test tube 50. In addition, when the test tube 50 is coupled to the test tube support structure 200, the catches 270a and 270b of the present disclosure may block the movement of the grip 210 to prevent the grip 210 from moving in the downward direction (-Y direction). Accordingly, when the test tube 50 is coupled to the test tube support structure 200, a shape of the grip 210 may be consistently maintained, and the structural reliability of the test tube support structure 200 can be improved.

In addition, the bottom fixing portion 280 of the present disclosure may protrude toward the groove 530H provided in the bottom 530 of the test tube 50 and may be inserted into the groove 530H. Accordingly, the bottom fixing portion 280 may safely fix the bottom 530 of the test tube 50.

FIG. 7 is a view illustrating a process in which the test tube 50 is separated from the box plate 100 according to the exemplary embodiment of the present disclosure.

Referring to FIG. 7, in order to separate the test tube 50 coupled to the box plate 100, the user may grip the grip 210 and move the grip 210 in a direction guided by the indication maker 290.

While the grip 210 is moved in the thickness direction (+Z direction) by a force of the user, the test tube lower portion support 220 and the first test tube side portion support 230 integrally provided with the grip 210 may also be moved in the thickness direction (+Z direction).

As the test tube lower portion support 220 and the first test tube side portion support 230 are moved, the lower portion and the side portion of the test tube 50 may also receive a force from the test tube lower portion support 220 and the first test tube side portion support 230 in the thickness direction (+Z direction). Accordingly, the test tube 50 may be easily separated from the box plate 100.

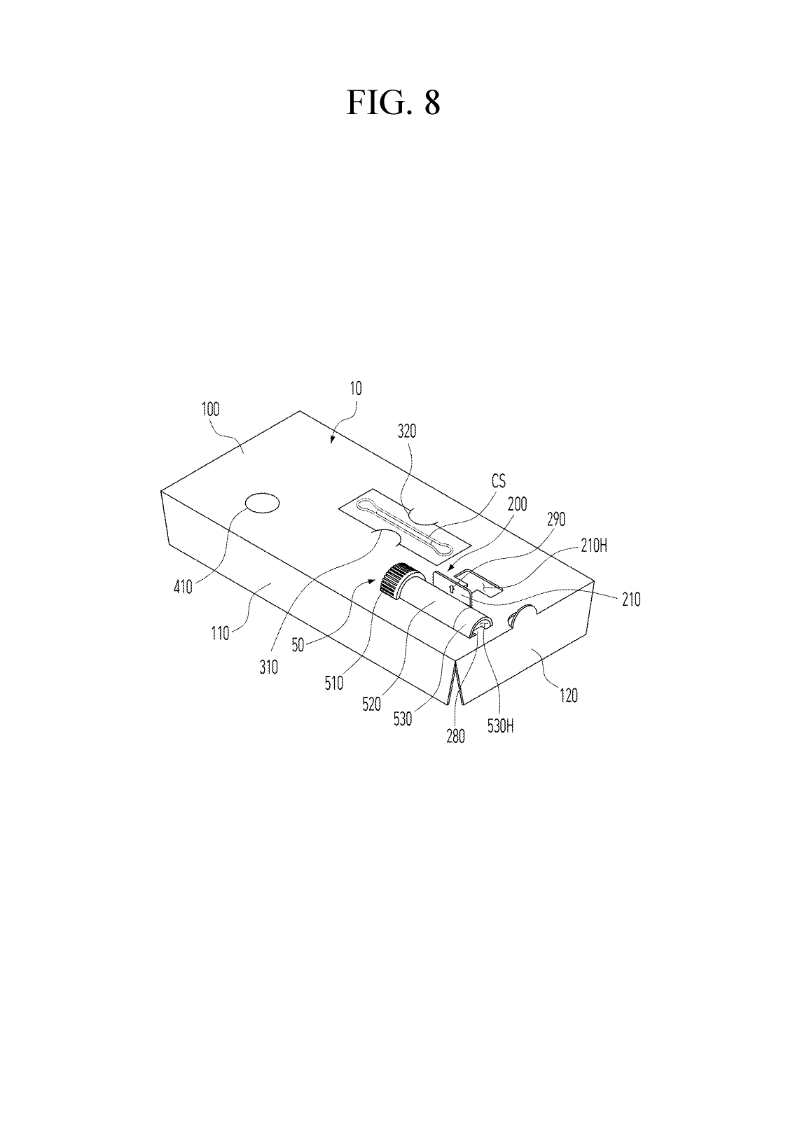

FIG. 8 is a perspective view illustrating the test kit box 10 according to the exemplary embodiment of the present disclosure.

Referring to FIG. 8, the test kit box 10 according to the exemplary embodiment of the present disclosure may further include the test swab support structures 310 and 320 and the test tube insertion member 410.

The test swab support structures 310 and 320 may be structures formed to support the test swab CS on the box plate 100. In the exemplary embodiment, at least portions of the test swab support structures 310 and 320 are separated from the box plate 100, and the test swab CS may be fixed on the basis of insertion coupling using the groove formed through the separation.

In the exemplary embodiment, the test swab support structures 310 and 320 may be provided as two structures having semicircular shapes symmetrical to each other. However, the shapes of the test swab support structures 310 and 320 are not limited to the above-described shapes.

The test tube insertion member 410 may be provided to allow the test tube to stand upright when a test is performed using the test tube 50. The test tube insertion member 410 may be provided by being cut with respect to the box plate 100. The test tube 50 may stand upright on the box plate 100 by being inserted into the hole which is the test tube insertion member 410 formed by being cut.

In the exemplary embodiment, the test tube insertion member 410 may be provided in a shape corresponding to the test tube 50. For example, when the test tube 50 is provided in a cylindrical shape, the test tube insertion member 410 may be provided in a circular shape, and when the test tube 50 is provided in a polygonal columnar shape, the test tube insertion member 410 may be provided in a polygonal shape.

While exemplary embodiments of the present invention have been described above in detail, the scope of the present invention is not limited thereto, and it should be understood that several modifications and improvements made by those skilled in the art using basic concepts described below in the appended claims are also included in the scope of the present invention.

What is claimed is:

1. A test kit box for accommodating a test tube, comprising:
a box plate; and
a test tube support structure of which at least a portion is separated from the box plate to support a test tube,
wherein the test tube support structure includes:
a grip separated from the box plate and provided on the box plate;
a test tube lower portion support extending from a portion of the grip and provided in a state in which at least a portion of the test tube lower portion support is folded under the box plate to support a lower portion of the test tube;
a first test tube side portion support of which one portion is connected to the test tube lower portion support, the other portion opposite to the one portion is connected to the box plate, and the other portion is folded beneath the box plate to support a first side portion of the test tube;
a second test tube side portion support provided in a side portion of the test tube lower portion support and folded beneath the box plate to support a second side portion opposite to the first side portion of the test tube; and
a catch extending from a folding line of the second test tube side portion support and separated from the box plate to protrude upward from the box plate to block movement of the grip,
wherein the grip is formed to move on the box plate through a hole formed by separating the test tube lower portion support when the test tube is coupled to the test tube support structure, and
wherein the catch restricts movement of the grip to prevent the grip from passing through the hole formed by separating the lower portion support and moving to a hole formed by separating the second test tube side portion support when the test tube is coupled to the test tube support structure.

2. The test kit box of claim 1, wherein the test tube support structure further includes:
a test tube lid support provided at one side of the first test tube side portion support and the second test tube side portion support and folded beneath the box plate to support a lid of the test tube; and
a test tube bottom support provided at the other side opposite to the one side of the first test tube side portion support and the second test tube side portion support and folded beneath the box plate to support a bottom of the test tube.

3. The test kit box of claim 2, wherein:
the first test tube side portion support is cut with respect to the box plate in a “⸦” shape rotated 90º clockwise;
the second test tube side portion support is provided as two second test tube side portion supports at both sides of the test tube lower portion support and is cut with respect to the box plate in a “⸦” shape rotated 90º counterclockwise;
the test tube lid support is cut with respect to the box plate in a “⸦” shape inverted in a left-right direction; and
the test tube bottom support is provided to have a smaller size than the test tube lid support and is cut with respect to the box plate in a “⸦” shape.

4. The test kit box of claim 3, wherein:
the test tube lower portion support and the second test tube side portion support are cut along a first shared cutting line extending in an up-down direction;
the test tube lid support, the first test tube side portion support, and the second test tube side portion support are cut along a second shared cutting line extending in the up-down direction;
the test tube bottom support, the first test tube side portion support, and the second test tube side portion support are cut along a third shared cutting line extending in the up-down direction; and
the first test tube side portion support and the second test tube side portion support are cut along a fourth shared cutting line extending in the left-right direction.

5. The test kit box of claim 2, wherein the test tube lower portion support is folded based on:
a first lower portion support folding line disposed to be collinear with a cutting line of the grip;
a second lower portion support folding line disposed under the first lower portion support folding line;
a third lower portion support folding line disposed to be collinear with the folding line of the second test tube side portion support; and
a fourth lower portion support folding line disposed to be collinear with a cutting line of the first test tube side portion support.

6. The test kit box of claim 2, further comprising a bottom fixing portion which extends from a folding line of the test tube bottom support and is inserted into a groove provided in the bottom of the test tube.

7. The test kit box of claim 1, wherein the grip includes an indication maker indicating a separation direction for separating the test tube.

8. The test kit box of claim 2, wherein a thickness of each of the grip, the test tube lower portion support, the first test tube side portion support, the second test tube side portion support, the test tube lid support, and the test tube bottom support is the same as a thickness of the box plate.
